SEQUENCE LISTING (4 sequences)

SEQ ID NO: 1 - DNA, 24 bp, Artificial Sequence
  The forward primer used for amplifing murine Oct-3/4 gene by PCR
ggcgttctctttggaaaggtgttc

SEQ ID NO: 2 - DNA, 20 bp, Artificial Sequence
  The reverse primer used for amplifing murine Oct-3/4 gene by PCR
ctcgaaccacatccttctct

SEQ ID NO: 3 - DNA, 22 bp, Artificial Sequence
  The forward primer used for amplifing murine GAPDH gene by PCR
ggtgaaggtcggtgtgaacgga

SEQ ID NO: 4 - DNA, 22 bp, Artificial Sequence
  The reverse primer used for amplifing murine GAPDH gene by PCR
tgttagtggggtctcgctcctg

(19) United States
(12) Patent Application Publication
(10) Pub. No.: US 2005/0164377 A1
(43) Pub. Date: Jul. 28, 2005

(54) Base material for culturing embryo stem cells and culture method

(76) Inventors: Tomoyuki Miyabayashi, Shizuoka (JP); Yoshihiro Hatanaka, Oita (JP)

Correspondence Address: BIRCH STEWART KOLASCH & BIRCH, PO BOX 747, FALLS CHURCH, VA, 22040-0747, US

(21) Appl. No.: 10/494,057

(22) PCT Filed: Oct. 31, 2002
(86) PCT No.: PCT/JP02/11388

(30) Foreign Application Priority Data
Oct. 31, 2001 (JP) 2001-334835

(51) Int. Cl. C12N 5/08

(52) U.S. Cl. 435/366; 435/404

(57) ABSTRACT

According to the present invention, capable of safely holding a large amount of undifferentiated embryonic stem cells to culture in the absence of feeder cells or feeder cell-derived components. Cultured embryonic stem cells can be applied to the fields of cell culture, tissue transplantation, drug development, and gene therapy.

### TECHNICAL FIELD

The present invention relates to a culture base material for embryonic stem cells, a culture method using the culture base material, and a culture apparatus using the culture base material. More specifically, the present invention provides a base material, a method, and an apparatus for culturing undifferentiated embryonic stem cells in the absence of feeder cells or feeder cell-derived components. The present invention can be applied to the fields of cell culture, tissue transplantation, drug development, and gene therapy.

### BACKGROUND ART

Internal organs and tissues that receive injury in the form of an externally caused wound, disease, aging, and the like must be regenerated to restore their functions. In particular, since the internal organs such as the heart, liver, kidney, and pancreas are indispensable to life-support, their functional decrease or abolition directly results in death. Medical transplantation is actively performed to save lives by organ transplant. However, a new approach is necessary for the solution due to the constant shortage of donors. Tissue transplants from a non-heart beating donor and heterotransplantation have been proposed as countermeasures. These methods, however, have serious problems. For example, the former has a problem of tissue preservation and the latter has problems of heterogenic immunity and pathogenic organ import. For this reason, the concept of regenerative medicine has come under close scrutiny in recent years. The concept of the term “regeneration” has already been known during the 20^{th} century relating to a means for increasing the regenerative power inherently possessed by individuals. The application of this concept to therapy has been undertaken. At the present time when the 21st century has been reached, however, the concept has been developed to the extent that the target is to produce tissues and organs to supplement lost organs by active utilization of stem cells, whereby the defects of organ transplant can be overcome. The specific target contemplated is to increase and differentiate stem cells having a growth capacity higher than functional cells. The differentiated stem cells are used for cell transplant or for artificial construction of organs together with artificial supporting tissues. The constructed organs are transplanted into living bodies and used as artificial internal organs. The problems with the conventional transplant treatment including autotransplantation such as a deficit of tissue or organs after their removal from a donor and shortage of donors are expected to be overcome, if the stem cells can actually be used for the treatment of the cell transplant and organ engineering.

Stem cells prospective in the application to such a treatment were discovered from experimental animals and humans and identified in a number of fields such as blood vessels, nerves, blood, cartilage, bone, liver, and pancreas. Among stem cells, the embryonic stem cells which are sometimes called pluripotent cells can differentiate into almost all cellular types and are expected to be used not only in the above-described reconstructive medical field, but also to be able to easily provide cells and tissues useful for drug development and gene therapy.

Embryonic stem cells (hereinafter referred to as “ES cells“) were discovered for the first time in mice as an established cell line that can be cultured in test tubes in an undifferentiated state while maintaining the differentiation potency into various individual formative tissues (Evans et al., Nature, 292, p 154, 1981). The culture potency of the ES cells can be maintained by forming normal embryos and chimera embryos while preserving the potency of differentiating into all adult mature cells. The ES cells can also produce various cells under in vitro differentiation induction conditions. Cells forming individuals are derived from the first epiblasts that deviate from embryoblasts (inner cell masses or ICM) or epiblasts in the blastocyst phase. In this sense, the embryoblasts (ICM) and epiblasts can be said to be stem cell groups possessing totipotency. The ES cells are cultured and separated from the ICM while maintaining the undifferentiated state.

ES cells have high differentiation potency and contribute to normal fetal generation by forming early embryos and chimera embryos in vivo. Mature cells originating from ES cells are detected in all internal organs of adults. For these reasons, the ES cells are deemed to be totipotent stem cells. On the other hand, ES cells can be differentiated into many cell groups such as blood cells, cardiac muscle cells, skeletal muscle cells, and nerve cells by operating the culture system in vitro.

In recent years, ES cell lines other than the mouse cells have been established. These ES cell lines were reported to have the same pluripotency as the mouse ES cells (cattle ES cells: Schellander et al., Theriogenology, 31, p 15-17, 1989; pig ES cells: Strojek et al., Theriogenology, 33, p 901, 1990; sheep ES cells: Handyside, Roux's Arch. Dev. Biol., 196, p 185, 1987; hamstar ES cells: Doetschman et al., Dev. Biol., 127, p 224, 1988; monkey ES cells: Thomson et al., Proc. Natl. Acad. Sci. USA, 92, p 7844, 1995; marmoset ES cells: Thomson et al., Biology of Production, 55, p 254, 1996; Human ES cells: Thomson et al., Science, 282, p 1145, 1998, Reubinoff et al., Nature Biotech, 18, p 399, 2000).

To maintain ES cells in an undifferentiated state, the ES cells must be cultured together with fibroblasts originating from normal fetuses as feeder cells. A similar method is used to maintain ES cell lines of the primate in an undifferentiated state (Thomson et al., Proc. Natl. Acad. Sci. USA, 92, p 7844, 1995, Thomson et al., Science, 282, p 1145, 1998, Reubinoff et al., Nature Biotech, 18, p 399, 2000).

In these ES cell culture methods using the feeder cells, however, the process for culturing the ES cell lines is complicated and time consuming. More recently, examples of endogenous virus infection among animals in different species have been reported (van der Laan et al., Nature, 407, p 90, 2000). Development of a culture method for medical application in which human ES cells are used while avoiding cell contact among animals of different species is, therefore, desired.

As a method for culturing ES cells while maintaining the undifferentiated state without feeder cells, a method using a culture dish coated with gelatin has been known. This method, however, requires addition of a leukemia inhibitory factor (LIF) to culture medium (Smith et al., Dev. Biol., 121, p 1, 1987), which involves high cost and difficult product quality control. The method, therefore, cannot be applied to large-scale production. In addition, the effect of LIF is limited to specific types of mice such as 129/sv and C57BL/6. A remarkable effect is not exhibited in other types of mice and animals in other species.

A method for culturing ES cells of primates without directly using feeder cells has also been reported (Japanese Patent Application Laid-open No. 2001-17163). However, since the addition of secretion components of fetal mouse fibroblasts to the culture medium is indispensable in this method, the problem of the above-mentioned endogenous virus remains unsolved.

Cell culture methods using culture media containing porous carriers have also been known (Japanese Patent Applications Laid-open No. 2001-120267, No. 2000-4870, and 2000-157261). These culture methods and culture base materials, however, can be only applied to specific types of differentiated cells. Neither a culture method nor culture base material that can culture embryonic stem cells while preserving the totipotency has been known heretofore.

### DISCLOSURE OF THE INVENTION

An object of the present invention is to provide a culture base material capable of safely holding a large amount of undifferentiated embryonic stem cells, a culture method for embryonic stem cells while maintaining embryonic stem cells in an undifferentiated state, and a culture apparatus.

When mouse embryo fibroblasts are used as the feeder cells, growth of undifferentiated multipotential embryonic stem cells can be thought to increase partly by the addition of the components produced by mouse embryo fibroblasts to the culture medium, whereas the feeder cell layer of the fibroblasts is considered to form a surface having a configuration and characteristics suitable for adhesion of embryonic stem cells. Contact of the embryonic stem cells with the feeder cell surface is predicted to stimulate the growth of the embryonic stem cells while being maintained in an undifferentiated state. Based on the above prediction, the inventors of the present invention have conducted extensive studies of several materials about the effect of maintaining the undifferentiated state of undifferentiated multifunctional embryonic stem cells. As a result, the inventors have found that the embryonic stem cells can be cultured while maintaining the undifferentiated state in the absence of feeder cells or feeder cell-derived components, if the similar surface conditions as the surface conditions of feeder cells are provided by using some materials and the embryonic stem cells are caused to come into contact with such a surface. This finding has led to the completion of the present invention.

Specifically, the present invention provides a base material capable of maintaining embryonic stem cells in an undifferentiated state in the absence of feeder cells or feeder cell-derived components. More specifically, the present invention provides a base material that is a porous material capable of maintaining embryonic stem cells in an undifferentiated state in the absence of feeder cells or feeder cell-derived components.

The present invention also provides a culture method capable of maintaining embryonic stem cells in an undifferentiated state in the absence of feeder cells or feeder cell-derived components. More specifically, the present invention provides a culture method using the above culture base material, in which the embryonic stem cells are maintained in an undifferentiated state in the absence of feeder cells or feeder cell-derived components.

The present invention further provides a method for capturing embryonic stem cells from a cell solution containing the embryonic stem cells using such an embryonic stem cell culture base material, and a cell capture material comprising such an embryonic stem cell culture base material capable of capturing the embryonic stem cells. Moreover, the present invention provides a culture apparatus for embryonic stem cells comprising a container packed with such a cell capture material.

### BRIEF DESCRIPTION OF THE DRAWINGS

FIG. 1 shows an image of ES cells cultured on the nonwoven fabric of Example 4 stained with alkaline phosphatase, wherein A-D indicates the following wells.
A: A well made of a plastic dish coated with gelatin
B: A well made of 0.03 denier nonwoven fabric coated with HM3
C: A well made of 0.014 denier nonwoven fabric coated with HM3
D: A well made of uncoated 0.014 denier nonwoven fabric

FIG. 2 shows Oct-3/4 gene expression amounts of ES cells cultured on. uncoated nonwoven fabrics of Example 5.

FIG. 3 shows an Oct-3/4 gene expression amount of ES cells cultured on a nonwoven fabric coated with HM3 of Example 5.

FIG. 4 shows an Oct-3/4 gene expression amount of ES cells cultured on a nonwoven fabric coated with HM3 and gelatin of Example 5.

FIG. 5 shows an Oct-3/4 gene expression amount of ES cells cultured on the PET nonwoven fabric with an average pore diameter of 12.0 μm of Example 7.

FIG. 6 shows Oct-3/4 gene expression amounts of ES cells cultured on nonwoven PET fabrics with an average pore diameter of 8.6 μm, 12.0 μm, or 13.4 μm, coated with gelatin of Example 7.

FIG. 7 shows Oct-3/4 gene expression amounts of ES cells cultured on cellulose nonwoven fabrics coated with gelatin.

FIG. 8 shows an alkaline phosphatase activity of ES cells cultured using micro carriers of Example 9.

### BEST MODE FOR CARRYING OUT THE INVENTION

The present invention will now be described in detail.

The present invention relates to a culture base material that can maintain undifferentiated embryonic stem cells as is, a culture method using the same, and a culture apparatus using the same. The culture base material, the culture method, and the culture apparatus of the present invention not only provide many advantages achievable by the use of the embryonic stem cells produced using the base material, the method, or the apparatus, but also can be used for the production of embryonic stem cells possessing one or more hereditary changes. Examples of the application include, but are not limited to, development of cell base models for diseases and development of tissues specified for transplantation used for the treatment of hereditary diseases.

Unless otherwise specified, the terms used herein have the meaning defined below. Unless otherwise defined, all other terms used in this specification have the meaning in accord with the definition in the specific field to which those terms relate.

Stem cells: stem cells indicate cells that can be differentiated into another cell type possessing specified functions (finally differentiated cells) or into another stem cell type that can be differentiated into another cell type of a smaller class.

Embryonic stem cells: embryonic stem cells are topipotent stem cells obtained from the morula of the embryo at the former embryo implantation stage or the blastocyst stage. Embryonic stem cells may also indicate multipotential stem cells originating from primordial germ cells of an embryo or fetus. These cells are also called EG cells. The embryonic stem cells used in the present invention may be those originating from any of the primates including humans, mammals, and birds.

Totipotency: the term totipotency is used relating to the cells that can be differentiated into arbitrary cell types including multipotential cells and completely differentiated cells (the cells that cannot be further differentiated into other cells).

Multipotency: the term multipotency is used relating to the cells that are not necessarily differentiated into all cell types, but can be differentiated into at least one of many different cell types. One example of a multipotent cell is the bone marrow stem cell that can be differentiated into not neurons but various blood cell types such as lymphocytes and erythrocytes. Therefore, all totipotent cells are multipotent, but all multipotent cells are not necessarily totipotent.

Undifferentiated: Undifferentiated means the state of any arbitrary cells possessing the potency of being differentiated into one or more cells that are in a more differentiated state.

Cell culture medium: the cell culture medium means a solution of salts and nutritive substances effective for sustaining growth of embryonic stem cells in a culture system.

Feeder cell: Feeder cells are non-embryonic stem cells on which embryonic stem cells are plated. The feeder cells provide the circumstance assisting the growth of plated embryonic stem cells.

Feeder cell origin components: The feeder cell origin components mean feeder cell crush components, including components secreted from feeder cells and cell membrane components. The leukemia inhibitory factor (LIF) is given as the component secreted from feeder cells. Extra-cellular matrices that are complexes composed of collagen, elastin, fibronectin, laminin, hyaluronan, chondroitin sulfate, dermatan sulfate, heparan sulfate, keratan sultate, and the like are also included.

Non-essential amino acids: non-essential amino acids indicate amino acids and include L-alanine, L-asparagine, L-asparatic acid, L-glutamic acid, glycine, L-proline, and L-serine.

The present invention provides a culture base material, a culture method, and a culture apparatus for growing and maintaining embryonic stem cells in an undifferentiated state. The culture base material and the method and apparatus using the same provided by the present invention can grow and maintain undifferentiated embryonic stem cells more simply and safely than conventional materials, methods, and apparatuses. The culture method for embryonic stem cells using the culture base material of the present invention can be used for screening specific differentiation inducing factors and useful combinations of two or more differentiation inducing factors. The capability of the culture base material and the culture method to proliferate totipotent embryonic stem cells in the undifferentiated state provides important advantages including production of embryonic stem cells that can be applied to therapeutic purposes.

The present invention provides a culture base material for growing and maintaining embryonic stem cells in an undifferentiated state. Specifically, a porous material can be used as the culture base material of the present invention.

The porous material is a base material having many minute holes that may be artificially formed. There are no specific limitations to the material, thickness, configuration, size, and the like of the porous material. Either organic or inorganic materials or composites of the organic material and inorganic material may be used for the porous material.

Among these, the organic materials, particularly organic polymer materials are preferable materials due to excellent processability such as cutting. Examples of the organic polymer compound that can be used as the porous material in the present invention include, but are not limited to, polyurethane, polyacrylonitrile, polyvinyl alcohol, polyvinyl acetal, polyester, polyamide, polystyrene, polysulfone, cellulose, cellulose acetate, polyethylene, polypropylene, polyvinyl fluoride, polyvinylidene fluoride, polytrifluorochlorovinyl, vinylidene fluoride-tetrafluoroethylene copolymer, polyether sulfone, poly(meth)acrylate, butadiene-acrylonitrile copolymer, polyether-polyamide block copolymer, ethylene-vinyl alcohol copolymer, and the like.

As examples of the inorganic material, silica materials such as glass and silicon wafer; ceramics such as alumina and zirconia; metals such as gold, silver, copper, iron, nickel, aluminum, and titanium; hydroxyapatite; and cement cured products can be given. As the composite material consisting of an organic material and inorganic material, a material in which nanosize particles of silica and organic material are dispersed, with either silica or the organic material being contained as major components, can be given, for example (Novak, M., Adv. Mater. 5, 422 (1993), Chujo, Y., Encyclp. Poly. Sci. Tech., CRC Press, Boca Raton, 6, 4793 (1996)).

There are no specific limitations to the shape of the porous material inasmuch as the material has pores that can support cells. The shape may be a plate, globe, rod, fibrous form, or hollow shape. Specific forms include a film, sheet, membrane, board, nonwoven fabric, filter paper, sponge, woven fabric, fabric, lump, thread, hollow yarn, and particles. Taking into account the factors such as easy and simple control of the pore size to support cells so that the cells are three-dimensionally cultured, easy fabrication of the culture base material, and the cost, the shape of the porous material is preferably a film, sheet, membrane, board, nonwoven fabric, sponge, hollow yarn, or particles, with particularly preferable shapes being particles and nonwoven fabric. There are no specific limitations to the pore size of the porous material. Taking into account the capability of three-dimensionally supporting the cells, an average pore size in the range of 0.1-150 μm is preferable, with a more preferable pore size being 1-50 μm. The particularly preferable pore size is 5-30 μm.

The average pore size as used in the present invention is measured and calculated by a mercury porosimeter, an optical microscope, or an SEM. When the average pore size is measured by a mercury porosimeter, the pore size at which the pore diameter distribution curve is the maximum peak is the average pore size in the present invention. When measured by observation using an optical microscope or an SEM, the configuration of the surface pore section is traced on a transparent film from the obtained image, an edge picture image of the pore section is prepared using an image scanner, and the circle-equivalent diameters of the opening (the pore section) is measured using an image analysis apparatus. The average pore size in the present invention is an average of 10 or more circle-equivalent diameters for such openings. Therefore, the average size indicates that particles with a diameter larger than the average pore size enter the pore of the porous material with difficulty, but this does not mean that particles having the larger diameter never enter the pores. In the present invention, the average pore size of a nonwoven fabric is a value measured using a mercury porosimeter and the average pore size of a micro carrier is a value determined by analyzing the observed image.

To the extent that the pores are not clogged, the surface of the porous material may be coated with a polymer compound to increase adhesion of cells thereto, the differentiation maintenance function, and the growth capability.

The polymer compound refers to a substance having a molecular weight of several hundred or more made from monomers having one or more recurring units that are linked one, two, or three dimensionally. The polymer compound can be broadly classified into three types, that is, natural polymers, semi-synthetic polymers, and synthetic polymers. As examples of the natural polymers, mica, asbestos, graphite, diamond, saccharides represented by starch, cellulose, and alginic acid; protein and peptide represented by gelatin, collagen, laminin, Vitronectin, fibronectin, and fibrinogen; and the like can be given. As examples of the semi-synthetic polymers, glass, nitrocellulose, cellulose acetate, hydrochlorinated rubber, carboxymethylcellulose, and the like can be given. As examples of the synthetic polymers, polyphosphonitrile chloride, polyethylene, polyvinyl chloride, polyamide, polyethylene terephthalate, polysulfone, polyacrylonitrile, polyvinyl alcohol, polymethyl methacrylate, polyhydroxyethyl methacrylate, polydimethylaminoethyl methacrylate, and copolymers made from two or more monomers such as a copolymer of hydroxyethyl methacrylate and dimethylaminoethyl methacrylate can be given.

Taking ease of coating treatment into consideration, organic polymers are preferable. Of the organic polymers, proteins, peptides, and organic synthetic polymers are preferable.

In addition, the porous material may be provided with some other surface treatments such as immobilization of a physiologically active substance such as LIF, grafting, radiation, and electron beams.

A method and apparatus that can separate embryonic stem cells from the group consisting of a plurality of different cells and can culture the captured embryonic stem cells in an undifferentiated state can be provided by using the culture base material consisting of the porous material of the present invention as a cell capture material.

Specifically, the method for culturing embryonic stem cells comprises introducing a cell solution containing embryonic stem cells and the cells to be removed into a container in which a cell capture material which is the culture base material of the present invention is filled, causing the embryonic stem cells to be captured by the cell capture material,, and culturing the embryonic stem cells in the container after the cells to be removed have been discharged from the container. The cell culture apparatus of the present invention is an apparatus in which a cell capture material which is the culture base material of the present invention is filled in a container, wherein the cell capture material can be used as a cell culture carrier and the container can be used for culturing the cells. The cells to be removed are all cells other than embryonic stem cells. Cells that have differentiated from the embryonic stem cells and lost the pluripotency are also included in the cells to be removed. Any cell-containing solution can be introduced into the cell capture material so long as the solution contains embryonic stem cells. Examples include-blood, marrow, debris tissue fluid, and a culture broth of stem cells and embryonic stem cells. The embryonic stem cells separated and cultured may be used as is or after suitable processing for cell transplant or as in various fields such as regenerative medical field, including tissue engineering.

The culture base material of the present invention can be used for an embryonic stem cell culture apparatus together with the cell culture medium described below. A nutrition blood serum or a blood-serum alternative may be added. Any optional cell culture medium can be used as a culture medium for the embryonic stem cells. Examples of the cell basic culture medium include, but are not limited to, Dulbecco-modified Eagle medium (DMEM), knock out MEM, Glasgow MEM (GMEM), RPMI1640, and IMDM (available from GIBCOBRL of the U.S.). A blood serum, a blood-serum alternative, or various growth factors may also be added to these basic media. The blood serum may be any optional blood serum or a solution of the blood serum that can supply a nutrient effective for the growth and maintenance of survivability of the embryonic stem cells. Examples of such a blood serum include fetal calf serum (FCS), cattle serum (CS), and horse serum (HS). An example of the blood-serum alternative includes, but is not limited to, knockout blood serum replacement (KSR, a product manufactured by GIBCOBRL, U.S.). In one embodiment, thebloodserumis fetal calf serum and the blood serum alternative is KSR. In a specific embodiment, the fetal calf serum or KSR is used in a concentration between about 1% and about 25%. In a more specific embodiment, the concentration of the fetal calf serum or KSR in a cell culture medium is 15%. Human-embryonic stem cells cultured in the culture medium containing a blood serum alternative using the culture base material of the present invention is very safe as compared with embryonic stem cells cultured by a conventional method due to non-contact with cells of different species or components derived from the cells of different species. The embryonic stem cells can be applied to clinical use such as cell transplant and tissue engineering.

The cell culture medium may also contain an anti-oxidant (a reducing agent, for example, β-mercaptoethanol). In a preferred embodiment, β-mercaptoethanol has a concentration of about 0.1 mMs. Other anti-oxidants (for example, monothioglycerol or dithiothreitol (DTT), or a combination of these) may be used to achieve the same effect. Furthermore, other equivalent substances are commonly known among persons skilled in the art in the field of cell culture.

The present invention further provides a culture base material for growing undifferentiated embryonic stem cells and a method for culturing cells in a medium containing this culture base material.

The embryonic stem cells to be cultured are available by using known methods and materials described below. Mouse ES cells: Evans et al., Nature, 292, p 154, 1981; cattle ES cells: Schellander et al., Theriogenology, 31, p 15-17, 1989; pig ES cells: Strojek et al., Theriogenology, 33, p 901, 1990; sheep ES cells: Handyside, Roux's Arch. Dev. Biol., 196, p 185, 1987; hamstar ES cells: Doetschman et al., Dev. Biol., 127, p 224, 1988; monkey ES cells: Thomson et al., Proc. Natl. Acad. Sci. USA, 92, p 7844, 1995; human ES cells: Thomson et al., Science, 282, p 1145, 1998, Reubinoff et al., Nature Biotech, 18, p 399, 2000). The mouse embryonic stem cells (129SV and C57/BL6) can be purchased from Dainippon Pharmaceutical Co., Ltd.

A more recent report describes that there are embryonic stem cells in the bone marrow, muscles, and brain of mice or human, in which an Oct-3/4 gene and Rex-1 gene which are markers specific to embryonic stem cells (Verfaillie et al., Nature Advance online publication, 20 Jun., 2002 (doi:10.1038/nature00870); Jiang et al., Experimental Hematology, 30, p 896, 2002). These reports suggest that totipotent cells equivalent to embryonic stem cells may also be present in an adult. As one of the features of the culture base material of the present invention, capability of maintaining expression of an Oct-3/4 gene of embryonic stem cells while maintaining undifferentiation can be given. Specifically, the adult origin stem cells similar to embryonic stem cells that express the Oct-3/4 gene may be cultured using the culture base material of the present invention while maintaining an undifferentiating state.

Once isolated, the embryonic stem cells can be cultured by any optional technique using the above cell culture medium and culture base material. For example, embryonic stem cells are disseminated on the sterilized porous material and the above-mentioned cell culture medium is added to culture the cells. A nonwoven fabric is given as an example of the porous material. The growth of embryonic stem cells is monitored to determine the degree of differentiation of the embryonic stem cells.

The degree of undifferentiation of the embryonic stem cells can be confirmed by measuring the amount of Oct-3/4 gene expression as described in Example 5. The Oct-3/4 gene is a transcription factor belonging to the POU family and is specifically expressed in the undifferentiated state in embryonic stem cells and embryonal carcinoma cells (EC cells) (Okamoto et al., Cell, 60, p 461, 1990). The gene is also expressed only by undifferentiated cell genealogy during embryonic growth (Scholer, Trend Genet, 7, p 323, 1991) and the expression is known to decrease as the differentiation proceeds. In addition, the Oct-3/4 gene has been found to play an important role in undifferentiated state maintenance due to the fact that homozygotes of Oct-3/4 gene-disrupted mice stop development during the blastocyst stage (Nichols et al., Cell, 95, p 379, 1998). The above-mentioned information gives suggestion that sustaining oct3/4 gene expression and/or inhibiting the reduction of oct3/4 gene expression means maintaining the undifferentiated state of the embryonic stem cells and inhibiting the differentiation of undifferentiated embryonic stem cells.

The quantitative PCR (polymerase chain reaction) method can be used as one means to measure the amount of expression of Oct-3/4 gene. Furthermore, the real-time PCR method that can ensure a simple and reliable determination in a broad dynamic range can be used. A method of using a TaqMan probe in which ABIPRISM77™ (Applied Biosystems) is used and a method of using LightCycler™ (Roche Diagnostic) are given as specific examples of the real-time PCR technology. Especially in the case of the latter method, the change in the amplification amount of DNA synthesized in each cycle in a high-speed reaction cycle, in which a temperature cycle is completed in several tens of minutes, can be detected. There are four methods for detecting DNA using the real-time PCR method: a method of using DNA-bonding coloring matter (Intercalator), a method of using a hybridization probe (Kissing probe), a method of using a TaqMan probe, and a method of using a Sunrise uni-primer (molecular beacon). It is also possible to analyze the amount of Oct-3/4 gene expression by using a DNA-bonding coloring matter such as SYBRGreen I. SYBRGreen I is a bonding coloring matter specifically bonding to the double stranded chains. Bonding to a double stranded chain reinforces its original fluorescence intensity. The PCR product can be detected, if the SYBRGreen I is added during the PCR reaction and the fluorescence intensity is measured at the end of each cycle of the extension reaction. To detect the Oct-3/4 gene, a primer is designed based on the sequence of Oct-3/4 gene using commercially available gene-analyzing software and the like in the same manner as in common PCR. An optimal primer must be produced. Otherwise, SYBRGreen I may detect non-specific products as well as specific products. As design criteria, consideration should be given to the length of oligomers, the base composition of the sequence, GC content, Tm value, and the like. To amplify the Oct-3/4 gene, a sense primer, OCT3 up: 5′-ggcgttctct ttggaaaggt gttc-3′ (Sequence ID No. 1 in Sequence Table) and an anti-sense primer, Oct3 down: 5′-ctcgaaccac atccttctct-3′ (Sequence ID No. 2 in Sequence Table) can be used. Although the oligonucleotide can be synthesized using a commercially available DNA synthesizer, it is possible to ask an expert to synthesize an oligomer of any optional sequence.

In many cases, the target of determination by the PCR method is the amount of the objective DNA in a given amount of sample. To this end, the amount of the sample first added to the reaction system must be evaluated. In this case, another DNA used as an internal standard reflecting the amount of the sample is measured separately from the objective DNA to correct the amount of the sample first added to the reaction system. A housekeeping gene that is deemed to exhibit no difference in the amount of expression by tissues and organs can be used as the internal standard to correct the amount of the sample. For example, glyceraldehydes triphosphate dehydrogenase (GAPDH) which is a major glycolysis enzyme; β-actin or γ-actin which is a component forming the cytoskeleton, and genes such as S26 which is a protein forming ribosome can be given.

The expression level of the Oct-3/4 gene is determined for the cells exposed to the culture base material of the present invention using the method described in the examples. The material increasing the amount of Oct-3/4 gene expression more than control cells differentiated from embryonic stem cells is deemed to be the culture base material which maintains the undifferentiated state of embryonic stem cells.

As another method for determining an optimized culture base material that can maintain the undifferentiated state of embryonic stem cells, a method of detecting an alkaline phosphatase (ALP) activity can be given. The ALP activity is known to be maintained in undifferentiated embryonic stem cells, but to decrease as the differentiation proceeds (Williams et al., Nature, 336, p 684, 1988, Thomson et al., Science, 282, p 1145, 1998). The ALP activity can be detected by cell staining using an insoluble substrate developing colors with ALP or by the ELISA (enzyme-linked immunosorbent assay) method using a water-soluble coloring substrate. As one embodiment, the ALP activity can be detected by cell staining using an alkaline phosphatase staining technique. Embryonic stem cells cultured in a cell culture medium using the porous material as the culture base material have been confirmed to contain an increased amount of undifferentiated cells as compared with the embryonic stem cells cultured using a cell culture dish made of plastic coated with gelatin by the ALP activity detection using this method.

As another method for screening the optimized culture base material maintaining an undifferentiated state of the embryonic stem cells, a method of detecting an antigen specifically expressed in undifferentiated cells such as stage specific embryonic antigens (SSEA) such as SSEA-1, SSEA-3, and SSEA-4 (Smith et al., Nature, 336, p 688, 1988, Solter et al., Proc. Natl. Acad. Sci. U.S.A, 75, p 5565, 1978, Kannagi et al., EMBO J.2, p 2355, 1983) can be given.

In one embodiment, the surface antigen such as SSEA-1 can be labeled by incubating with the specific antibody (primary antibody) that recognizes this antigen, and further incubating with the second antibody (secondary antibody) bonded with a reporter such as a fluorescence label. This procedure can cause the cells expressing the target antigen to become fluorescent. The labeled cells can be counted using a standard method, for example, a method of using a flow site meter, and can further be separated. Next, the numbers of labeled and unlabeled cells can be compared to determine the effect of the objective culture base material. Alternatively, after being exposed to a non-labeled cell surface marker antibody, the cells can be exposed to a second specific antibody to an anti-cell surface antigen antibody (for example, anti-SSEA-1 antibody) in an ELISA (enzyme-linked immunosorbent assay) type, whereby the number of the cells expressing a desired surface antigen can be determined calorimetrically or by measuring fluorescence. Other methods to determine cells exhibiting the surface antigen are commonly known among persons skilled in the art of cell culture.

The culture base material and the culture method exhibiting improved performance in the growth of undifferentiated embryonic stem cells can be expected to be applied to all technologies in which the embryonic stem cells are useful.

The cells produced by using the culture base material and the culture method for the present invention can be differentiated and the differentiated cells are used for cell transplant or for artificial construction of organs together with artificial supporting tissues. The constructed organs are transplanted into living bodies or used as artificial internal organs. The application of the stem cells to a cell transplant treatment and tissue engineering can solve the problems with conventional autotransplantation such as a deficit of tissue or organs after their removal from a donor and shortage of donors.

The culture base material and culture method for the present invention are used for the production of embryonic stem cells having a single or two or more hereditary alterations. Hereditary alterations of the cells is desirable for many reasons, for example, providing cells modified for a gene therapy and substitute tissues for replant (to avoid rejection of cells by a host). According to the present invention, the amount of embryonic stem cells can be increased using the above-described culture base material and culture method. The first gene is altered in or introduced into at least one of the cells in a cell culture product. A first clone group of the altered embryonic stem cells is induced from the resulting culture product. The first clone group can be grown using the culture base material of the present invention to establish a cell line possessing a desired hereditary alteration. If further alteration is required, a second gene is altered in or introduced into at least one of the cells in the first clone group, whereby a second clone group of cells possessing the first and second hereditary alterations is produced. Alternatively, it is possible to introduce the first and second hereditary alterations into the same embryonic stem cells to screen both alterations at the same time, thereby avoiding the necessity of isolating the first clone group. However, a stepwise procedure is more preferable.

Any optional method for producing a hereditary transformant known in the technical field of molecular biology can be used to hereditarily alter the cells. Such a method includes, but is not limited to, a method of using a positive-negative selective vector described below (U.S. Pat. No. 5,464,764, U.S. Pat. No. 5,487,992, U.S. Pat. No. 5,627,059, and U.S. Pat. No. 5,631,153 to Capecchi et al.).

Furthermore, a yeast artificial chromosome (YAC) can be used for the hereditary alteration described below (U.S. Pat. No. 5,981,175). Another method that can be included is a method described in U.S. Pat. No. 5,591,625 to Gerson et al. that relates to preparation of stem cells that may increase expression of specific gene products, signaling molecules, cell surface proteins, and the like for a medical treatment application of the prepared stem cells. These patents are incorporated as a whole in this specification as references for all purposes.

As clear for a person skilled in the art, expression of altered gene products can be achieved by alteration of the coding sequence of the gene product or alteration of the adjoining region of the coding sequence. Therefore, the term “hereditary alteration” used in the present specification includes the alteration of the sequence encoding the gene product and the alteration in the adjoining region, especially the alteration in the 5′ upstream side of the coding sequence (including the promoter). Similarly, the term “gene” includes a coding sequence, regulatory sequences which may be present adjoining with the coding sequence, and other sequences adjoining the coding sequence. In addition, as known in the field concerned, a hereditary alteration may be attained by introducing a nucleic acid which does not necessarily include all genome sequences into cells (for example, by introducing a nucleic acid which may be inserted into a genome by recombination).

The cells cultured and/or altered using the culture base material and the culture method for the present invention may be attached to the support surface to screen substances with biological activity. The cells are bonded to a substrate so that intracellular electrophysiology changes in response to external stimulus may be measured for use as a means for screening a high process amount of biologically active substances. The cells may be transformed by a specific gene therein or by DNA that labels, expresses, or knockouts the gene product. Many compounds can be quickly and precisely screened by combining the cells attached to chips in this manner with a measuring device such as a computer, for example. A biosensor can also be arranged and combined with measuring for large-scale parallel screening.

Moreover, a reporter gene can be incorporated into DNA of the embryonic stem cells functionally combined with a copy of a gene relevant to specific disease conditions by using the method mentioned above. The reporter is sensitive to both a transcriptional phenomenon and a post-transcriptional phenomenon. The stem cells can differentiate in such a manner that each differentiated descendant may contain one copy of disease gene and reporter structure, respectively. Subsequently, these cells are screened in regard to a presumed medical treatment factor. In this manner, it is possible to correlate the gene expression and the response to a potential medical treatment factor with the differentiation state of the cells. This type of screening strategy may be implemented using the above-mentioned high processing biosensor according to appropriate selection of the reporter. Other applications of the biosensor of the type described in the present specification are clear to a person skilled in the art.

Determination of the Oct-3/4 gene expression level as a marker for the embryonic stem cell differentiation described in Example 5 can be used to determine the degree of undifferentiation of the embryonic stem cells cultured using the culture base material and the culture method for the present invention. The embryonic stem cells cultured using the culture base material and the culture method for the present invention are differentiated and used for cell transplant or for constructing artificial organ. The cells may be either hereditarily unaltered or hereditarily altered using the above-described method. The pluripotent cells identified as highly expressing the Oct-3/4 gene maybe specifically isolated and may be used for cell transplant or for further culture and/or alteration as mentioned above.

Furthermore, the use of the culture base material and the cellular method of the present invention for providing a culture product of altered or unaltered embryonic stem cells may be applied to monitoring embryonic stem cells or screening substances that can improve collection of the stem cells. For example, a presumed embryonic stem cell amplifing substance may be added to the cell culture product proliferated using the above-mentioned method. As compared with a control cell culture product deficient of a presumed embryonic stem cell amplification factor, a substance that increases Oct-3/4 gene expression to a certain level is identified as an embryonic stem cell amplification factor.

### EXAMPLES

Examples will now be described. These examples illustrate only one of the embodiments of the present invention and are not intended to limit the scope of the present invention.

### Example 1

Preparation of Embryonic Stem Cell Culture Medium

To proliferate embryonic stem cells, an ES cell culture medium was prepared by adding the following factors to Dulbecco's Modified Eagle Medium (hereinafter referred to as DMEM, Cat. No. 11995, manufactured by GIBCO BRL Co.) at final concentrations shown below. 15% fetal calf serum (manufactured by BIO WHITTAKER), 0.1 mM β-mercaptoethanol (manufactured by SIGMA), 1× nonessential amino acid stock (Cat. No. 11140-050 manufactured by GIBCO BRL Co.), 1 mM sodium pyruvate (Cat. No. 11360-070 manufactured by GIBCO BRL Co.), 2 mM L-Glutamine (Cat. No. 25030-081 manufactured by GIBCO BRL Co.), and 1000 units/ml ESGRO (manufactured by CHEMICON International Inc. (product number ESG1107): containing mouse LIF as an active ingredient). An ES cell assay culture medium for ES cell differentiation suppression assay was prepared by removing ESGRO from this ES cell culture medium.

### Example 2

Culture of Embryonic Stem Cells

Gelatin (Type A: from porcine SKIN, G2500 manufactured by SIGMA Co.), was dissolved in distilled water to a concentration of 0.1%, was sterilized. A dish for cell culture with a diameter of 6 cm was coated to 5 ml of a sterilized 0.1% aqueous solution of gelatin, and allowed to stand at room temperature for 10 minutes or more. The aqueous solution of gelatin was removed. 2×10⁶ mouse embryo primary culture cell (Cat. No. YE9284400 manufactured by Lifetech Oriental Co.) treated with mitomycin C (manufactured by KYOWA HAKKO KOGYO Co., Ltd.) was disseminated and cultured for 5 hours or more at 37° C. in 5 ml of DMEM containing 10% fetal bovine serum (manufactured by GIBCO BRL) using a 5% CO₂ incubator (manufactured by Tabai Espec Corp.). D3ES cells of mouse embryonic stem cell line (available from Rolf Kemler, Max Planck Institut fur Immunbiologie, Stuheweg 51, D-79108 Freiburg, Germany) were disseminated over the feeder layer of mouse embryo primary cultured cells (fibroblast cells). The cells were cultured and proliferated for two days at 37° C. in 5 ml of ES culture medium using a 5% CO₂ incubator.

### Example 3

ES Cell Differentiation Suppression Assay

The D3ES cells cultured in Example 2 were washed twice with PBS. After the addition of 0.25% trypsin solution (15090-046 manufactured by GIBCO BRL), the mixture was incubated for 5 minutes at 37° C. Undifferentiated D3ES cell colonies were removed from the feeder. 5 ml of ES cell culture medium was added, the cell colonies were distributed using a small pipette, moved to a 15 ml sterilized tube, and centrifuged for 5 minutes at 800 rpm using a desktop centrifuge (manufactured by TOMY SEIKO Co., Ltd.) to pelletize the cells. The supernatant was removed. The cells were suspended again in 5 ml of a fresh ES cell culture medium, disseminated on a cell culture dish with a diameter of 6 cm previously coated with a 0.1% aqueous solution of gelatin, and incubated for 20 minutes at 37° C. After 20 minutes, the medium containing floating cells was collected and moved to a sterilized tube using a pipette and pelleted by centrifugation for 5 minutes at 800 rpm using a desktop centrifuge. The supernatant was removed and the cells were suspended again in 5 ml of ES cell assay medium. A nonwoven fabric was placed on the bottom of a 6-well cell culture dish (Cat. No. 3046 manufactured by FALCON). 1×10⁴ cells were disseminated on the nonwoven fabric and cultured for 7 days in 3 ml of ES cell assay culture medium. A nonwoven fabric made from PET (polyethylene terephthalate) as a base material with a fineness of 0.014 denier and an average pore size of 10 μm (or a fineness of 0.03 denier and an average pore size of 13 μm) coated with a 12% ethanol solution of a 97:3 copolymer of HM-3 (2-hydroxyethyl)methacrylate (hereafter abbreviated as HEMA) and N,N-dimethyl methacrylate (hereafter abbreviated as DM) was used as the nonwoven fabric for ES cell culture. An uncoated nonwoven fabric was also used.

### Example 4

Alkaline Phosphatase Staining

ES cells were stained using an alkaline phosphatase kit (Cat. No. 86-R manufactured by SIGMA Diagnostic Co.). The culture medium was removed by suction from each well in which the ES cells were cultured as described in Example 3. The cells were washed one time with 2 ml of a phosphate buffered physiological saline solution (PBS). After the addition of 2 ml of a cell fixing fluid (25 ml of citric acid solution (Cat. No. 91-5 manufactured by SIGMA), and 65 ml of acetone, 8 ml of 37% formaldehyde) to each well, the dish was allowed to stand for 30 seconds at room temperature. The fixing fluid was removed by suction and 2 ml of deionized water was added to each well. The dish was allowed to stand for 45 seconds at room temperature. The deionized water was removed by suction and alkaline phosphatase staining solution (1 ml of sodium nitrite solution, 1 ml of first red violet LB salt solution, 1 ml of naphtol AS-BI alkali solution, 45 ml distilled water) was added in an amount of 2 ml/well. The dish was allowed to stand for 15 minutes at room temperature. After removing the staining solution by suction, the wells were washed with 2 ml of deionized water. The stained image of each well is shown in FIG. 1. Wells (B-D) on which a nonwoven fabric was laid exhibited a significantly high alkaline phosphatase activity as compared with the gelatin coated well (A) as a control. Specifically, the nonwoven fabric was proven to be effective for maintaining ES cells in an undifferentiated state.

### Example 5

Quantitative Determination of the Amount of Oct-3/4 Gene Expression.

The amount of Oct-3/4 gene expression of ES cells was measured using a Light Cycler (manufactured by Roche Diagnostics). The total RNA was extracted from the ES cell cultured by the method shown in Example 3 using the SV total RNA isolation system (manufactured by Promega Corporation) according to the method described in the attached protocol. cDNA was synthesized using the resulting total RNA as a template and using an Oligo (dT) 12-18 primer (18418-012 manufactured by GIBCO BRL) and the Omni script reverse transcriptase (manufactured by Qiagen Co.) according to the attached protocol. PCR was carried out using 2 μl among the 20 μl of synthesized cDNA as a template and using A Light Cycler First Start DNA master SYBRGreenI kit (manufactured by Roche Diagnostics) according to the attached protocol. The expression amounts of the Oct-3/4 gene and glyceroaldehyde triphosphate dehydrogenase (GAPDH) gene as a control were measured. A sense primer, OCT3 up: 5′-ggcgttctct ttggaaaggt gttc-3′ (Sequence ID No. 1 in Sequence Table) and an anti-sense primer, Oct3 down: 5′-ctcgaaccac atccttctct-3′ (Sequence ID No. 2, Sequence Table) were used to amplify the Oct-3/4 gene. A sense primer, GAPDH up: 5′-ggtgaaggtc ggtgtgaacg ga-3′ (Sequence ID No. 3 in Sequence Table) and an anti-sense primer, GAPDH down: 5′-tgttagtggg gtctcgctcc tg-3′ (Sequence ID No. 4, Sequence Table) were used to amplify the GAPDH gene. The composition of the PCR reaction solution and the reaction conditions are shown in Table 1 and Table 2, respectively.

TABLE 1
Composition of PCR reaction solution for Oct-3/4 gene
determination

| | |
|---|---|
| 25 mM MgCl₂ | 1.6 |
| 10 μM Sense Primer | 0.5 (Final conc. 0.25 μm) |
| 10 μM Antisense Primer | 0.5 (Final conc. 0.25 μm) |
| CDNA | 2.0 |
| 10 × LC-DNA Master | 2.0 |
| H₂O | 13.4 |
| Total | 20.0 μl |

TABLE 2
PCR reaction conditions for Oct-3/4 gene determination

| | | | | | | Rate of temp. |
|---|---|---|---|---|---|---|
| | | Cycle | | Temp | Time | change |
| Step | Program | No. | Fraction | (° C.) | (sec) | (° C./sec) |
| 1 | Denaturation | 1 | 1 | 95 | 600 | 20 |
| 2 | Amplification | 45 | 1 | 95 | 15 | 20 |
| | | | 2 | 55 | 10 | 20 |
| | | | 3 | 72 | 20 | 20 |
| 3 | Melting curve | 1 | 1 | 95 | 0 | 20 |
| | analysis | | 2 | 65 | 10 | 1 |
| | | | 3 | 95 | 0 | 0.1 |

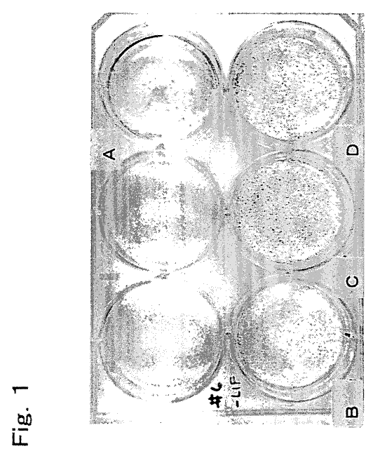

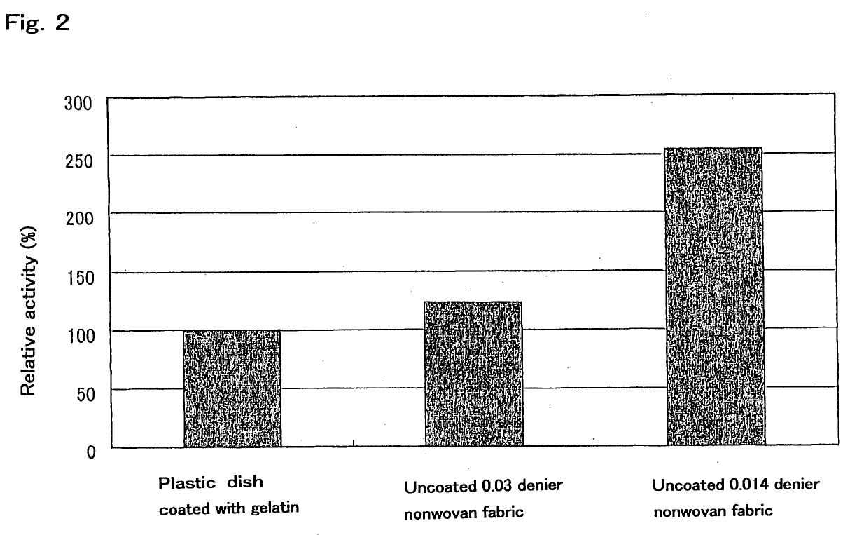

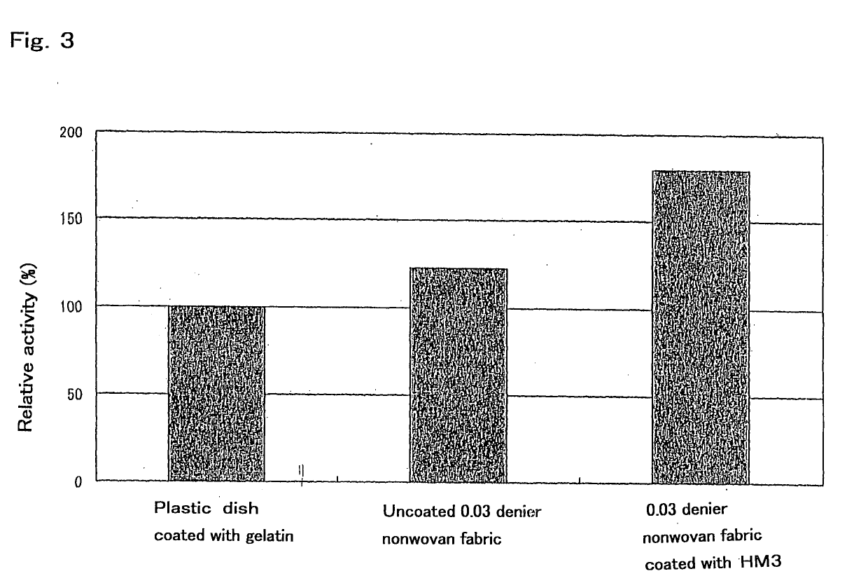

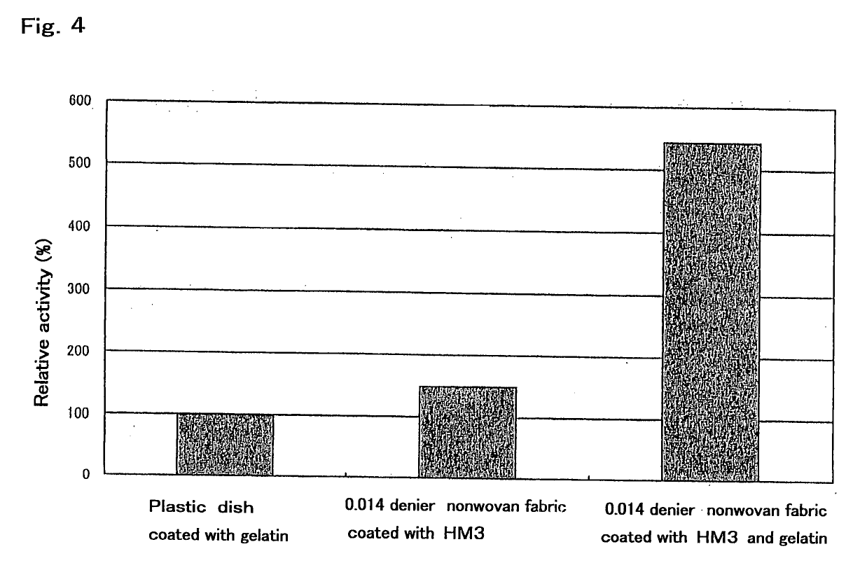

Oct-3/4 gene expression of ES cells cultured on the nonwoven fabric was significantly accelerated as compared with ES cells cultured on gelatin. The effect was particularly remarkable on nonwoven fabric with a fineness smaller than 0.03 deniers (FIG. 2). The same acceleration effect of Oct-3/4 expression was seen also on nonwoven fabric coated with HM3 polymer (FIG. 3). Moreover, expression of Oct-3/4 was remarkably accelerated on an HM3-coated nonwoven fabric of which the surface was previously impregnated with a 0.1% aqueous solution of gelatin (FIG. 4). From the above result, the nonwoven fabric was also shown to maintain ES cells in an undifferentiated state from the amount of Oct-3/4 expression.

### Example 6

ES Cell Differentiation Suppression Assay 2

The D3ES cells cultured in Example 2 were washed twice with PBS. After the addition of 0.25% trypsin solution (15090-046 manufactured by GIBCO BRL), the mixture was incubated for 5 minutes at 37° C. Undifferentiated D3ES cell colonies were removed from the feeder. 5 ml of ES cell culture medium was added, the cell colonies were distributed using a small pipette, moved to a 15 ml sterilized tube, and centrifuged for 5 minutes at 800 rpm using a desktop centrifuge (manufactured by TOMY SEIKO Co., Ltd.) to pellet the cells. The supernatant was removed. The cells were suspended again in 5 ml of a fresh ES cell culture medium, disseminated on a cell culture dish with a diameter of 15 cm previously coated with a 0.1% aqueous solution of gelatin, and incubated for 20 minutes at 37° C. After 20 minutes, the medium containing floating cells was collected using a pipette, disseminated again in a cell culture dish with a diameter of 15 cm coated with a 0.1% aqueous solution of gelatin, and incubated for 20 minutes at 37° C. After 20 minutes, the medium containing floating cells was collected and moved to a sterilized tube using a pipette and pelleted by centrifugation for 5 minutes at 800 rpm using a desktop centrifuge. The supernatant was removed and the cells were suspended again in 5 ml of ES cell assay medium. A nonwoven fabric cut into a circle with a diameter of 20 mm was placed on the bottom of a 12-well cell culture dish (Cat. No. 3043 manufactured by FALCON) and 1 ml of an ES cell assay culture medium was added. 1 ml of a cell solution prepared to a concentration of 5×10³ cells/ml in ES cell assay culture medium was disseminated to culture the cells for 7 days. The nonwoven fabric laid on a 12-well cell culture dish was previously impregnated with PBS for 10 minutes and then with an ES assay culture medium for 10 minutes or more. With the nonwoven fabric coated with gelatin, a nonwoven fabric was previously impregnated with PBS for 10 minutes, with 0.1% aqueous solution of gelatin for 10 minutes, and then with an ES assay culture-medium for 10 minutes or more.

As the nonwoven fabric for ES cell culture, nonwoven fabrics made from PET as a base material having an average pore size of 8.6 μm (fiber diameter: 1.15 μm), 12.0 μm (fiber diameter: 1.2 μm), or 13.4 μm (fiber diameter: 1.7 μm) (all manufactured by Asahi Kasei Corporation) were used. As nonwoven fabrics made from a cellulose base material, Bemliese™ #PS140 (average pore size: 47 μm), Bemliese™ #TS327 (average pore size: 113.8 μm), and Bemliese™ #SF184 (average pore size: 114.1 μm) (all manufactured by Asahi Kasei Corporation) were used.

### Example 7

Quantitative Determination of the Amount of Oct-3/4 Gene Expression 2

The amount of Oct-3/4 gene expression of ES cells was measured using A Light Cycler (manufactured by Roche Diagnostics). The total RNA was extracted from the ES cell cultured by the method shown in Example 6 using the ISOGEN (manufactured by Nippon Gene Co., Ltd.) according to the method described in the attached manual. Specifically, after removing the culture medium from the dish after culture and washing with 2 ml of PBS, ISOGEN was added in an amount of 1 ml/well. The mixture was allowed to stand for 5 minutes at room temperature and transferred into a 1.5 ml Eppendorf tube. After the addition of 0.2 ml of chloroform (manufactured by Wako Pure Chemical Industries, Ltd.), the mixture was shaken for 15 seconds, allowed to stand for 2-3 minutes, and centrifuged using a small quantity centrifuge (TOMY SEIKO Co., Ltd.) at 14,000×g for 15 minutes at 4° C. 400 μl of the supernatant was transferred to a new Eppendorf tube. After the addition of 500 μl of isopropanol (manufactured by Wako Pure Chemical Industries, Ltd.), the mixture was allowed to stand for 10 minutes at room temperature and centrifuged using the small quantity centrifuge at 14,000×g for 10 minutes at 4° C. After removing the supernatant, 1 ml of 70% ethanol was added. The mixture was shaken and centrifuged using the small quantity centrifuge at 10,000×g for 5 minutes at 4° C. The supernatant was removed. The precipitate was dried and dissolved in 30 μl distilled water to obtain the total RNA solution. cDNA was synthesized using the resulting total RNA as a template and using deoxyribonucleaseI (amplification grade, Invitrogen), an Oligo (dT) 12-18 primer (18418-012 manufactured by GIBCO BRL) and the Omni script reverse transcriptase (manufactured by Qiagen Co.) according to the attached protocol. Specifically, a reaction solution was prepared by adding 1 μl of 10× DNaseI Reaction Buffer and 1 μl of 10× DNaseI (both manufactured by Invitrogen) to 1 μg of the total RNA, and adding distilled water to the mixture to make the total amount 10 μl. The reaction solution was incubated for 15 minutes at room temperature. After the addition of 1 μl of 25 mM EDTA, the reaction solution was heated for 10 minutes at 65° C. The reaction solution was allowed to cool to room temperature. After the addition of 2 μl of 10× Buffer RT, 2 μl of 5 mM dNTP Mix, 2 μl of Oligo(dT) 12-18 primer, 0.25 μl of RNaseOUT (GIBCO BRL, Cat. No. 10777-019), and 1 μl of Omniscript Reserve Transcriptase, the total amount was made 20 μl with the addition of Rnase-free distilled water. The mixture was incubated at 37° C. for 60 minutes to obtain a cDNA solution. A part of the synthetic cDNA obtained in this manner was diluted with distilled water to 5-fold. PCR was carried out using 2 μl of the thus-diluted cDNA as a template and using A Light Cycler First Start DNA master SYBR GreenI kit (manufactured by Roche Diagnostics) according to the attached protocol. The expression amounts of the Oct-3/4 gene and glyceroaldehyde triphosphate dehydrogenase (GAPDH) gene as an internal standard were measured. A sense primer, OCT3 up (Sequence ID No. 1 in Sequence Table) and an anti-sense primer, Oct3 down (Sequence ID No. 2, Sequence Table) were used to amplify the Oct-3/4 gene. A sense primer, GAPDH up (Sequence ID No. 3 in Sequence Table) and an anti-sense primer, GAPDH down (Sequence ID No. 4, Sequence Table) were used to amplify the GAPDH gene. The composition of the PCR reaction solution and the reaction conditions are shown in Table 3 and Table 4, respectively.

TABLE 3
Composition of PCR reaction solution for Oct-3/4 gene
determination

| | |
|---|---|
| 25 μM MgCl₂ | 1.6 |
| 10 μM Sense Primer | 0.5 (Final conc. 0.25 μm) |
| 10 μM Antisense Primer | 0.5 (Final conc. 0.25 μm) |
| cDNA (5-fold dilution) | 2.0 |
| 10 × LC-DNA Master | 2.0 |
| H₂O | 13.4 |
| Total | 20.0 μl |

TABLE 4
PCR reaction conditions for Oct-3/4 gene determination

| | | | | | | Rate of temp. |
|---|---|---|---|---|---|---|
| | | Cycle | | Temp. | Time | change |
| Step | Program | No. | Fraction | (° C.) | (sec) | (° C./sec) |
| 1 | Denaturation | 1 | 1 | 95 | 600 | 20 |
| 2 | Amplification | 45 | 1 | 95 | 15 | 20 |
| | | | 2 | 55 | 10 | 20 |
| | | | 3 | 72 | 20 | 20 |
| 3 | Melting curve | 1 | 1 | 95 | 0 | 20 |
| | analysis | | 2 | 65 | 10 | 1 |
| | | | 3 | 95 | 0 | 0.1 |

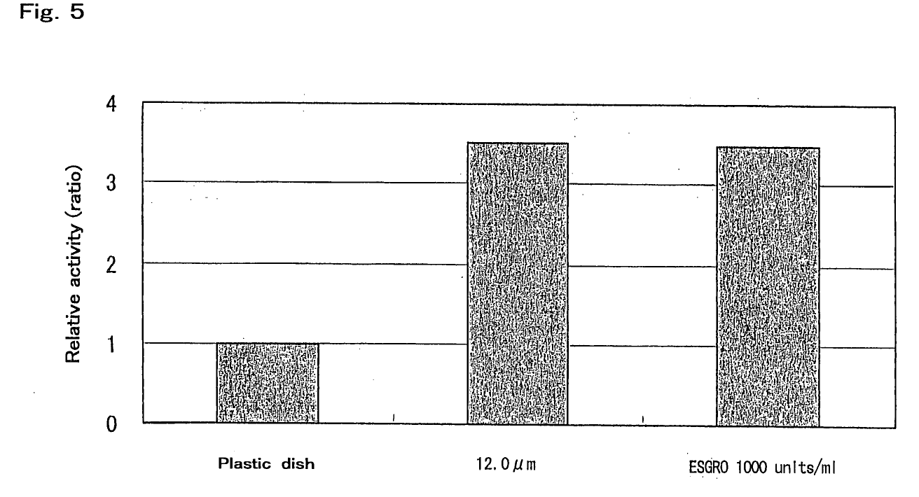

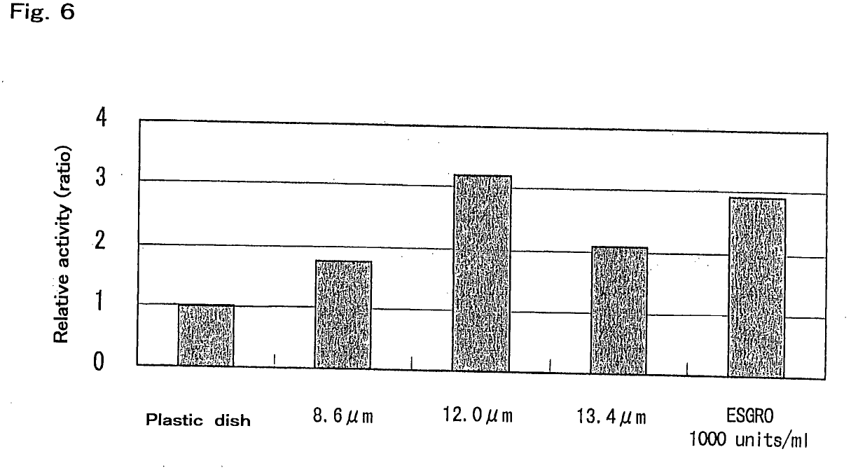

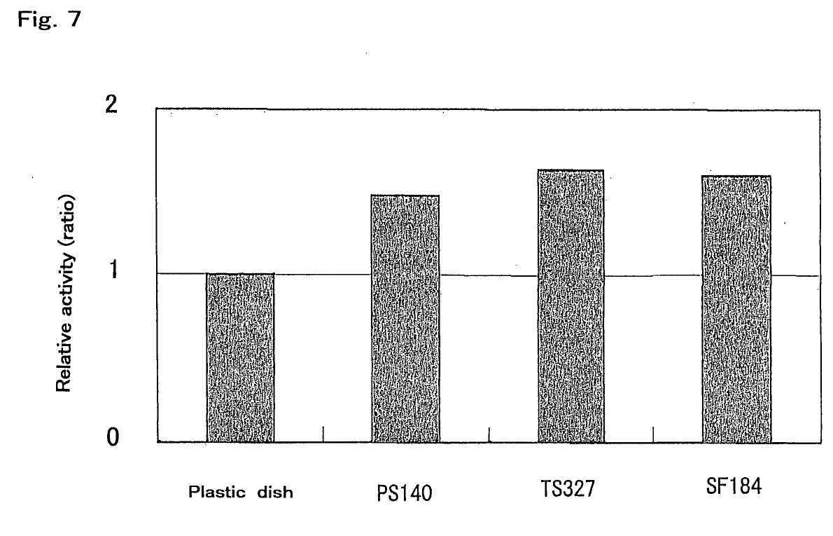

Oct-3/4 gene expression of ES cells cultured on the nonwoven PET fabric was significantly accelerated as compared with the ES cells cultured on a plastic dish. The effect of maintaining the Oct-3/4 gene expression on the nonwoven fabric with an average pore size of 12.0 μm was confirmed to be equivalent to that on ESGRO 1000 unit/ml (FIG. 5). The same effect was confirmed when the plastic dish and nonwoven fabric were impregnated with gelatin (FIG. 6). Specifically, from the amount of Oct-3/4 gene expression the nonwoven PET fabric was confirmed to maintain undifferentiated conditions of the ES cells. In addition, the nonwoven fabric with an average pore size of 12 μm was confirmed to be most effective. In the same manner as in the case of the nonwoven PET fabric, it was confirmed that a cellulose nonwoven fabric can also maintain the oct3/4 gene expression (FIG. 7). From the above result, the nonwoven fabric was also shown to maintain ES cells in an undifferentiated state from the amount of Oct-3/4 gene expression.

### Example 8

ES Cell Differentiation Suppression Assay 3

The D3ES cells cultured in Example 2 were washed twice with PBS. After the addition of 0.25% trypsin solution (15090-046 manufactured by GIBCO BRL of the U.S.), the mixture was incubated for 5 minutes at37° C. Undifferentiated D3ES cell colonies were removed from the feeder. 5 ml of ES cell culture medium was added, the cell colonies were dispersed using a pipette with a small diameter, moved to a 15 ml sterilized tube, and centrifuged for about 5 minutes at 800 rpm using a desktop centrifuge (manufactured by TOMY SEIKO Co., Ltd.) to pellet the cells. The supernatant was removed. The cells were suspended again in 5 ml of a fresh ES cell culture medium, disseminated on a cell culture dish with a diameter of 15 cm previously coated with a 0.1% aqueous solution of gelatin, and incubated for 20 minutes at 37° C. After 20 minutes, the medium containing floating cells was collected using a pipette, disseminated again in a cell culture dish with a diameter of 15 cm coated with a 0.1% aqueous solution of gelatin, and incubated for 20 minutes at 37° C. After 20 minutes, the medium containing floating cells was collected and moved to a 15 ml sterilized tube using a pipette and pelleted by centrifugation for 5 minutes at 800 rpm using a desktop centrifuge. The supernatant was removed and the cells were suspended again in 5 ml of ES cell assay medium. After the addition of 0.5 ml of Asahi Kasei micro carriers (average pore size: 30 μm, manufactured by Asahi Kasei Corporation, Japan) to a 24-well cell culture dish (Cat. No. 3047 manufactured by FALCON, U.S.), an ES cell solution prepared in suspending cells in an ES cell culture medium at a concentration of 2×10³ cells/ml or 1×10⁴ cells/ml was charged in an amount of 0.5 ml/well. The cells were cultured for 7 days at 37° C. in a 5% CO₂ atmosphere. The sample containing ESGRO was prepared by previously adding ESGRO to the cell solution to a final concentration of 1,000 units/ml. Micro carriers were suspended in PBS, sterilized for 20 minutes under pressure while heating at 121° C., replaced by an ES cell culture medium, and suspended in an ES cell culture medium of the amount 4 times the bed volume. Micro carriers coated with gelatin were sterilized under pressure, replaced by a 0.1% aqueous solution of gelatin, and suspended in an ES cell culture medium of an amount 4 times the bed volume.

### Example 9

Quantitative Determination of Alkaline Phosphatase

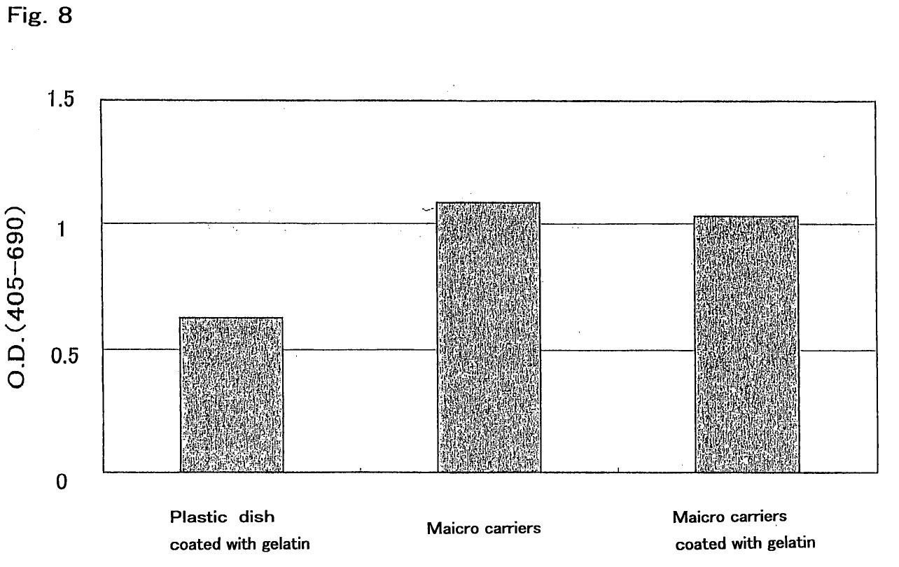

The alkaline phosphatase activity of the ES cells was determined using a p-nitrophenyl phosphate solution (Product No. NPPD-1000, manufactured by MOSS Inc. of the U.S., hereinafter referred to as p-NPP). The culture medium was removed by suction from each well in which the ES cells were cultured as described in Example 8. The cells were washed three times with 1 ml of a phosphate buffered physiological saline solution (PBS). After the addition of 200 μl of p-NPP to each well, the mixture was allowed to stand for 10 minutes at room temperature. 25 μl of 8 M sodium hydroxide solution was added to each well to terminate the reaction. 100 μl of the reaction solution was charged to a 96-well micro test plate (Cat. No. 3072 manufactured by FALCON of the U.S.). The absorbencies of the solution at 405 nm (O.D.₄₀₅) and at 690 nm (O.D.₆₉₀) were measured using an absorbance meter (SPECTRA MAX190 manufactured by Molecular Devices, Inc.) to determine the alkaline phosphatase activity as the difference (O.D.₄₀₅-O.D.₆₉₀) of the absorbencies. The results are shown in the graph of FIG. 8. The alkaline phosphatase activity of the ES cells cultured using micro carriers was significantly higher than the alkaline phosphatase activity of the ES cells cultured without using the micro carriers. The micro carrier thus supported undifferentiated ES cell culture.

### INDUSTRIAL APPLICABILITY

According to the present invention, embryonic stem cells in an undifferentiated state can be cultured in a large amount and in a safe manner in the absence of feeder cells or feeder cell-derived components. The embryonic stem cells obtained are useful and can be applied to the fields of cell culture, tissue transplantation, drug development, gene therapy, and the like.

1. A culture base material for embryonic stem cells comprising a porous material.

2. The culture base material according to claim 1, wherein the culture base material can maintain the embryonic stem cells in an undifferentiated state.

3. The culture base material according to claim 1 or 2, wherein the culture base material comprises neither feeder cells nor feeder cell-derived components.

4. The culture base material according to claim 1, wherein the porous material is a nonwoven fabric.

5. The culture base material according to claim 1, wherein the porous material is coated with a polymer compound.

6. The culture base material according to claim 1, wherein the porous material has an average pore size of 0.1-150 μm.

7. A method for culturing embryonic stem cells comprising using the culture base material according to claim 1 to culture the embryonic stem cells while maintaining the cells in an undifferentiated state.

8. A method for capturing embryonic stem cells comprising using the culture base material according to claim 1 to capture the embryonic stem cells from a cell solution containing the embryonic stem cells.

9. A cell capturing material for embryonic stem cells comprising the culture base material according to claim 1.

10. An embryonic stem cell culture apparatus comprising the cell capturing material according to claim 9 packed in a container.
